(19) United States
(12) Patent Application Publication
(10) Pub. No.: US 2004/0235128 A1
(43) Pub. Date: Nov. 25, 2004

(54) Process for the preparation of deactivated rice bran lipase

(75) Inventors: Raghavendra Manikanahally Papannegowda, Mysore (IN); Prakash Vishveshwaraiah, Mysore (IN)

Correspondence Address: Ladas & Parry, 26 West 61st Street, New York, NY, 10023, US

(73) Assignee: COUNCIL OF SCIENTIFIC AND INDUSTRIAL RESEARCH

(21) Appl. No.: 10/818,118

(22) Filed: Apr. 5, 2004

Related U.S. Application Data
(63) Continuation of application No. 09/820,189, filed on Mar. 28, 2001, now abandoned.

(51) Int. Cl. C12N 9/99

(52) U.S. Cl. 435/184

(57) ABSTRACT

The present invention provides a process for the preparation of deactivated rice bran lipase by

a) extracting lipase enzyme from rice bran and purifying the said lipase enzyme using a salting out agent to obtain active lipase enzyme;

b) preparing a ligand in the ratio of 1:10, 1: 100, 1:250, 1: 750 and 1:1500 mole to mole ratio of protein to ligand;

c) mixing the said active lipase enzyme and the ligand and adding to it a substrate, followed by the addition of an activator such as CaCl₂ in a concentration of 0.1 M;

d) incubating the mixture thus obtained for 4 hours to check for activity,

e) separating the deactivated lipase enzyme from the mixture.

### FIELD OF THE INVENTION

The present invention relates to a process for the preparation of deactivated rice bran lipase. More particularly, the present invention relates to a process for the preparation of deactivated rice bran lipase using benzene boronic acid.

### BACKGROUND OF THE INVENTION

The demand for enzymes is ever increasing owing to their applications in a wide variety of processes. Enzymes find use in a large number of fields such as nutrition, food science and clinical medicine as well. In the above areas, lipases have attracted a great deal of attention in recent years.

Lipases, belong to the class of hydrolases, catalyze the cleavage of ester bonds in tri-, di- and monoglycerides. The natural substrates of lipases are triglycerides having very low solubility in water. Today, lipases are the enzymes of choice for organic chemists, pharmacists, biochemists, biotechnologists, microbiologists, food technologists and biochemical engineers.

Enzymes such as lipase, which are undesirable in food systems, need to be deactivated and hence an improved method of stabilization of rice bran for the production of food grade rice bran oil is necessary. Rice bran contains 10-26% of edible quality oil but large scale and successful harnessing of this source to produce edible grade oil has been hampered by the high lipase content of the bran. Many physical and chemical methods have been tested to minimize lipase activity in rice bran with varying levels of success. Stabilization of rice bran, in other words, inactivation of rice bran lipase by chemical inhibitors, especially competitive, would be expected to augment the country's demand for the edible oil supply.

The effect of several proteins such as melittin, β-lactoglobulin A, serum albumin, ovalbumin and myoglobin were tested to inactivate pancreatic lipase and microbial lipase. This inhibition could be the result of desorption of lipase from its substrate due to a change in interfacial quality (Gargouri, Y, et al 1984. Inhibition of pancreatic and microbial lipases by proteins. Biochim. Biophys. Acta., 795, 326-331).

The bile salt dependent lipase from human milk catalyzes the hydrolysis of the water-soluble and water insoluble substrates and is competitively inhibited by phenyl boronic acid and it binds near or at the active site serine with 15 fold. Therefore phenyl boronic acid bears analogy to a substrate rather than to a tetrahedral intermediate analog (Abouakil, N and Lombardo, D. 1989. Inhibition of human milk bile salt—dependent lipase by boronic acids. Implication to the bile salts activator effect. Biochim. Biophys. Acta. 1004, 215-220).

In porcine pancreatic lipase, the inhibition by octadecane boronic acid was competitive when measured against the hydrolysis of dissolved tripropionin in the presence of siloconized glass beads. Boronic acids were believed to be analogs of the tetrahedral intermediate in the action of lipase (Gamer, C. W. 1980. Boronic acid inhibitors of porcine pancreatic lipase. J. Biol. Chem., 255(11), 5064-5068.

The study of inhibition of lipoprotein lipase by benzene boronic acid indicates the presence of serine and histidine in the active site of the enzyme. This inhibition is believed to be due to the formation of an inhibitor-enzyme complex, The presence of apolipoprotein C-II reverses the inhibition of lipoprotein lipase (Vainio, P, Virtanen, J. A and kinnunen, P. K. J. 1982. Inhibition of lipoprotein lipase by benzene boronic acid. Effect of apolipoprotein C-II. Biochim.Biophys.Acta. 711, 386-390).

The lipoprotein lipase which hydrolyzes triglycerides in lipoproteins and in synthetic emulsions decreases sharply with the amount of products formed unless albumin is present. This was due to the direct interaction of the enzyme with fatty acids and loss of the lipolysis-stimulating effects of activator proteins (Bengtsson, G. and Olivecrona, T. 1980. Lipoprotein lipase. Mechanism of product inhibition. Eur.J.Biochem. 106, 557-562).

While a large body of information is available on the inhibition of enzymes in general and lipase in particular, information on the inhibition of plant lipases is scanty.

Reference is made to Bremer, Klaus-Dieter, Sawlewicz and Pavel (2000), Taiwan Patent 381025B where a lipase inhibitor such as tctrahydrolipstatin, lipstatin, valilactone, esterastin, ebelactone A and ebelactone B were used as active substances and normal pharmaceutical carriers.

Reference is made to Takahashi and Hidehiko (1996), U.S. Pat. No. 5,503,831 wherein a composition having lipase inhibiting activity is prepared by extracting defatted rice germ with water at room temperature, which is useful in preventing obesity and can be incorporated into food products.

Reference is made to Stoddart, Barry and Narinx, Emmanuel (1999) WIPO Patent 9948471A1 Wherein use of copolymeric compounds of average molecular weight of atleast 400 having a polyalkoxy backbone, comprising atleast one branched alkoxy unit and at least one linear alkoxy unit for the preparation of compounds for the reduction of the lipase activity in particular of microorganisms, reduction of skin rash or irritation, dandruff formation and malodour of the body and for the preservation of food and beverage products is described.

Reference is made to Uchino Keijiro, Mizuno Takashi and Kawaguchi Kiyomi (1997), Japanese Patent 9040689A2 wherein inhibitor is obtained by blending a compound of triterpenes and its derivatives such as their salts or acetylated substances comprising oleanolic acid, ursonic acid, their salts and acetylated substances as an active ingredient for inhibiting activities against the lipase and capable of preventing a food containing oil and a fat from deteriorating due to the lipase and reducing the calorie of the food and useful even for prevention of adult diseases.

Reference is made to Kawaguchi Kiyomi Mizuno Takashi and Uchino Keijiro (1996), Japanese Patent 8268882A2 wherein using an inhibitor containing hinokitiol as an active ingredient capable of manifesting excellent activities against lipases having high safety and useful for prevention from the deterioration of a food due to the lipases in the food containing oils and fats and prophylaxis etc., of adult diseases. The daily dose for an adult is preferably 0.5-3000 mug and the amount of blended active ingredient is 0.3-15 wt % in the case of peroral or mucosal absorption and 0.01-10 wt % in the case of parenteral administration respectively. But when the inhibitor is blended in a food, it is preferably used in an amount of 0.001-10 wt %.

The main draw back of all above methods is the use of irreversible inhibitors to inactivate the enzyme activity to prevent deterioration of food due to the lipase.

### OBJECTS OF THE INVENTION

The main object of the present invention is to provide a process of inactivation of lipase before the material is processed to obtain edible grade oil.

Another object of the present invention is to provide higher stability of rice bran.

### SUMMARY OF THE INVENTION

Accordingly the present invention provides a process for the preparation of deactivated rice bran lipase, which comprises:

a). extracting lipase enzyme from rice bran and purifying the said lipase enzyme using a salting out agent to obtain active lipase enzyme;

b). preparing a ligand in the ratio of 1:10, 1: 100, 1:250, 1: 750 and 1: 1500 mole to mole ratio of protein to ligand;

c). mixing the said active lipase enzyme and the ligand and adding to it a substrate, followed by the addition of an activator such as CaCl₂ in a concentration of 0.1 M;

d). incubating the mixture thus obtained for 4 hours to check for activity,

e). separating the deactivated lipase enzyme from the mixture.

In one embodiment of the invention, the salting out agent is selected from ammonium sulfate and CaCl₂.

In a further embodiment of the invention, the purification of the lipase enzyme in step (a) of the process is done by dialysis and size-exclusion chromatography.

In another embodiment of the invention, the substrate is selected from triacetin and tributyrin.

In a further embodiment of the invention, the mixture of the active lipase enzyme and the ligand is added to the substrate at a concentration of at least 5%.

In another embodiment of the invention, the ligand used comprises an aromatic boro compound.

In yet another embodiment of the invention, the lipase enzyme is mixed with the ligand in a ratio 1:10, 1: 100, 1:250, 1: 750 and 1:1500 on a mole to mole ratio of protein to ligand.

### DETAILED DESCRIPTION OF THE INVENTION

The different unit operations and conditions involved in the preparation of lipase solution in presence of ligands is given in the reaction schemes given below:

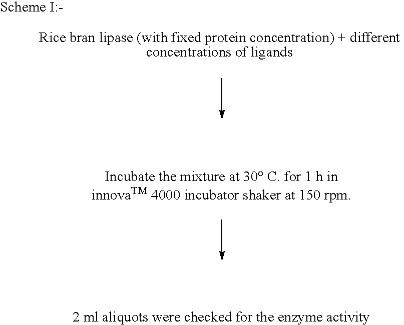

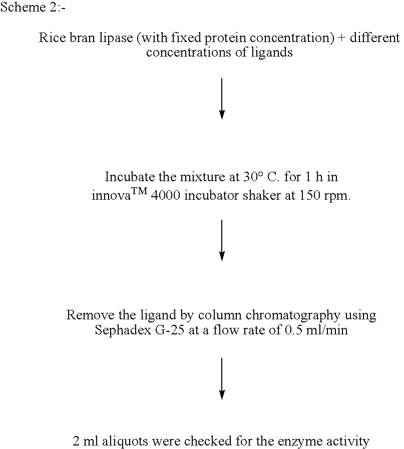

To approximately 2 mg of rice bran lipase in 0.05 M sodium phosphate buffer, pH 7.4 is added different concentrations of ligands ranging from 1:10 to 1:1500 mole to mole ratio of protein to ligand and incubated for 1 h. at 30° C. in Innova™ 4000 incubator shaker at 150 rpm. The reaction mixture was checked for enzyme activity by pH-stat method using Mettler Toledo DL12 titrator using 5% solution of triacetin or tributyrin as substrate. Respective blank solutions where the enzyme was inactivated by the addition of distilled alcohol was also used. The activity was expressed in terms of control activity as microequivalents of alkali consumed per mg of protein per hour.

To approximately 2 mg of rice bran lipase in 0.05 m sodium phosphate buffer, pH 7.4 is added different concentrations of ligand ranging from 1:10 to 1:1500 on a mole to mole ratio and incubated fpr 1 hr at 30° C. in Innova™ 4000 incubator shaker at 150 rpm. The reaction mixture was checked for enzyme activity by pH start method using mettler Toledo DL 12 titrator using 5% solution of triacetin or tributyrin as substrate. Respective blank solutions where the enzyme was inactivated by the addition of distilled alcohol was also used. The activity was expressed in terms of control activity as micro equivalent of alkali consumed per mg of protein per hour.

The enzyme solution containing different concentrations of ligands was passed through a sephadex G-25 column which was previously equilibrated with the elution buffer. The protein eluting immediately after the void volume was collected and assayed for the activity.

The novelty and the uniqueness of the process of the invention stems from the reversibility of the enzyme activity due to deactivation of rice bran lipase using benzene boronic acid.

The following examples are given by way of illustration of the present invention and therefore should not be construed to limit the scope of the present invention

### EXAMPLE-1

| CONSTITUENTS | QUANTITY |
|---|---|
| Lipase concentration | 2 mg/ml |
| Ligand in the reaction mixture(mole/mole) | 1:10 |
| Substrate (5%, w/v) | 4 ml |
| CaCl₂ (0.1 M) | 10 μl |

In order to understand the effect of inhibitors on the activity of rice bran lipase experiments were carried out at different concentrations of ligands. The activity of rice bran lipase in presence of 1: 10 mole to mole ratio of protein to ligand was measured. The analysis of the data showed that the enzyme was found to loss 35% of initial activity in presence of 1:10 mole to mole ratio of protein to ligand.

### EXAMPLE-2

| CONSTITUENTS | OUANTITY |
|---|---|
| Lipase concentration | 2 mg/ml |
| Ligand in the reaction mixture(mole/mole) | 1:1500 |
| Substrate (5%, w/v) | 4 ml |
| CaCl₂ (0.1 M) | 10 μl |

The activity of rice bran lipase was measured in presence of 1: 1500 mole/mole and the data showed that the enzyme losts nearly 77% of its initial activity. The enzyme was also checked for reversibility of its original activity after removal of ligands by gel filtrations on Sephadex G-25 column.

### EXAMPLE-3

| CONSTITUENTS | QUANTITY |
|---|---|
| Lipase Concentration | 2 mg/ml |
| Ligand in the reaction | 1:10 |
| Mixture (mole/mole) | |
| Substrate (5% w/v) | 4 ml |
| Cacl₂ (0.1 M) | 10 μl |

The reversibility was checked by using Sephadex G-25 column chromatography in the presence of 1:10 ratio. The results indicated that the enzyme was able to recover the initial activity.

### EXAMPLE-4

| CONSTITUENTS | QUANTITY |
|---|---|
| Lipase Concentration | 2 mg/ml |
| Ligand in the reaction | 1:1500 |
| Mixture (mole/mole) | |
| Substrate (5% w/v) | 4 ml |
| Cacl₂ (0.1 M) | 10 μl |

In the presence of 1:1500 mole to mole ratio, enzyme recovered almost its original activity after the removal of ligand by using gel filtration on Sephadex G-25 column.

The main advantages of the present invention are:

(1) The deactivation of rice bran lipase is brought up by using specific reversible inhibitors, it can target on the active site of the enzyme than inactivation.

(2) The concentrations of inhibitor needed is very low.

(3) The process of deactivation is reversible with the removal of inhibitor.

(4) The other properties of lipase such as its functional attributes doesn't get altered other than activity.

We claim:

1. A process for the preparation of deactivated rice bran lipase, which comprises:
a) extracting lipase enzyme from rice bran and purifying the said lipase enzyme using a salting out agent to obtain active lipase enzyme;
b) preparing a ligand in the ratio of 1: 10, 1: 100, 1:250, 1: 750 and 1:1500 mole to mole ratio of protein to ligand;
c) mixing the said active lipase enzyme and the ligand and adding to it a substrate, followed by the addition of an activator such as CaCl₂ in a concentration of 0.1 M;
d) incubating the mixture thus obtained for 4 hours to check for activity,
e) separating the deactivated lipase enzyme from the mixture.

2. A process as claimed in claim 1 wherein the salting out agent is selected from ammonium sulfate and CaCl₂.

3. A process as claimed in claim 1 wherein the purification of the lipase enzyme in step (a) of the process is done by dialysis and size-exclusion chromatography.

4. A process as claimed in claim 1 wherein the substrate is selected from triacetin and tributyrin.

5. A process as claimed in claim 1 wherein the mixture of the active lipase enzyme and the ligand is added to the substrate at a concentration of at least 5%.

6. A process as claimed in claim 1 wherein the ligand used comprises an aromatic boro compound.

7. A process as claimed in claim 6 wherein the aromatic bromo compound is benzene boronic acid.

8. A process as claimed in claim 1 wherein the lipase enzyme is mixed with the ligand in a ratio 1: 10, 1: 100, 1:250, 1: 750 and 1: 1500 on a mole to mole ratio of protein to ligand.
